(19) United States
(12) Patent Application Publication
(10) Pub. No.: US 2024/0201178 A1
(43) Pub. Date: Jun. 20, 2024

(54) DETECTION ASSAY

(71) Applicants: RANDOX LABORATORIES LTD., Crumlin Antrim (GB); RANDOX TEORANTA, County Donegal Dunglos (IE)

(72) Inventors: Peter Fitzgerald, Crumlin Antrim (GB); John Victor Lamont, Crumlin Antrim (GB); Ivan McConnell, Crumlin Antrim (GB); Ciaran Richardson, County Donegal Dungloe (IE); Philip Lowry, Crumlin Antrim (GB); Jim Curry, Crumlin Antrim (GB)

(21) Appl. No.: 18/284,783

(22) PCT Filed: Mar. 31, 2022
(86) PCT No.: PCT/EP2022/058703

§ 371 Date: Sep. 28, 2023

(30) Foreign Application Priority Data
Mar. 31, 2021 (EP) 21166455.2
Mar. 31, 2021 (GB) 2104662.8
Nov. 9, 2021 (GB) 2116054.4

(51) Int. Cl. G01N 33/543; G01N 33/68

(52) U.S. Cl. CPC G01N 33/54353; G01N 33/54393; G01N 33/6857; G01N 2650/00

(57) ABSTRACT

The present invention relates to improved detection assays via the utilisation of a substrate with an analyte immobilised on its surface by means of a linking agent and a cross-linker. The present invention also relates to a method of producing a substrate for use in detection assays and a method of detecting an analyte using said substrate.

### FIELD OF THE INVENTION

The present invention relates to improved detection assays that utilise a substrate with an analyte immobilised on its surface. The present invention also relates to a method of producing a substrate for use in detection assays and a method of detecting an analyte using said substrate.

### BACKGROUND OF THE INVENTION

The use of detection assays to detect and characterise analytes in a given sample is well known. For example, arrays of polynucleotides are widely used in DNA sequencing procedures and in hybridisation studies for the detection of genetic variations in a patient. Immunoassays are also well known for detecting analytes, such as specific proteins or other binding agents, through their properties as antigens or antibodies.

Detection assays typically comprise a supporting material comprising a plurality of reaction zones located in spatially distinct areas on the substrate onto which a binding agent is immobilized. Such a configuration allows for simultaneous testing of multiple analytes or biomarkers in a sample, if so desired. The use of detection assays in both the detection and measurement of analytes are an important and widely used laboratory tool that can be used as a predictive/diagnostic tool in a wide range of clinical diseases and/or infections.

However, despite the widespread use of such detection assays, there is still scope for, and a continuing need for, rapid detection assays with improved accuracy and precision that can be easily adapted for detection of a wide range of analytes, and consequently, a wide range of clinical diseases and/or infections. This is particularly relevant in view of the current SARS-CoV-2 coronavirus pandemic, in which the importance of diagnostic testing is of paramount importance in the continuing effort to control the outbreak of a novel virus. As such, detection assays that are highly sensitive, efficacious, and have the versatility to display these advantageous properties whilst detecting a broad range of analytes would be highly desirable.

### SUMMARY OF THE INVENTION

The present invention is based on the surprising discovery that the use of a particular cross-linker to aid immobilisation of an analyte to a substrate support provides improvements in the performance of an assay. Additionally, said cross-linker may be used for the detection of a broad range of analytes, demonstrating the versatility of said assay as a predictive/diagnostic tool across a range of clinical diseases/infections.

Accordingly, in a first aspect, the present invention provides for a substrate comprising a plurality of discrete reaction zones onto which one or more biological molecules are immobilised to the substrate surface by means of a linking agent and a cross-linker, wherein the cross-linker is a sulphone-based cross linker having a structure according to Formula (1):

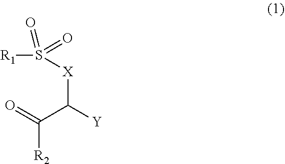

wherein X is —CH₂—;
Y is methylidene (═CH₂), or Y is selected from —CH₂—SO₂-Ph-R₄, —CH₂—SO₂-Ph-CH₃, —CH₂—SO₂—CH₃, —CH₂—O—SO₂-Ph-R₄, —CH₂—O—SO₂-Ph-CH₃ or —CH₂—O—SO₂—CH₃; preferably Y is methlylidene (═CH₂), —CH₂—SO₂-Ph-CH₃, or —CH₂—O—SO₂-Ph-CH₃, more preferably Y is methylidene (═CH₂) or —CH₂—SO₂-Ph-CH₃;
R₁ is CH₃ or CH₃-Ph, wherein the Ph is optionally substituted with one or more C₁₋₆alkyl, NO₂, F, Cl or Br; preferably R₁ is CH₃-Ph, and Ph is substituted with a C₁₋₆alkyl group, preferably methyl; and
R₂ is selected from —C₆₋₁₂aryl-Z, —C₁₋₁₈alkyl-Z, and —C₂₋₂₀alkenyl-Z, wherein Z is selected from COR₃, —NH₂ and —OH, R₃ is selected from H, OH, NH₂, —C₁₋₆alkyl-OH and (EtO)₃Si—(CH₂)ₙ—NH— in which n is 1-6; and R₄ is H or is optionally substituted with one or more C₁₋₆alkyl, NO₂, F, Cl or Br; preferably R₂ is selected from -Ph-Z, —C₁₀H₈—Z, —C₁₋₆alkyl-Z and —C₂₋₆alkenyl-Z, more preferably from -Ph-Z and —C₁₀H₈—Z, and more preferably -Ph-Z, and preferably Z is selected from COR₃.

In a second aspect, the present invention provides for a method for producing a substrate having a biological molecule immobilised thereon, comprising attaching the biological molecule to the substrate surface by means of a sulphone-based cross-linker having a structure according to Formula (1).

In a third aspect, the present invention provides for a method of detecting the presence of an analyte in a sample obtained from a subject wherein said analyte has affinity for a biological molecule, or portion thereof, and wherein said method comprises bringing the sample obtained from the subject into contact with the biological molecule, or portion thereof, which is immobilised on a substrate support, detecting the binding of the analyte to the biological molecule, or portion thereof, via means of a detectably-labelled molecule, and measuring the amount of binding of said analyte compared to a control wherein the biological molecule, or portion thereof, is immobilised to the substrate by means of a linking agent and a cross-linker, wherein the cross-linker is a sulphone-based cross-linker having a structure according to Formula (1).

### DESCRIPTION OF FIGURES

FIG. 1 shows the cross-linker 4-[bis(4-methylphenylsulphonylmethyl)-1-oxoethyl] benzoic acid, which can incorporate a silane moiety prior to addition to the substrate (see FIG. 7, substrate linking agent B) or can be bonded to a substrate-linking agent (such as a silane moiety) already present on the substrate surface.

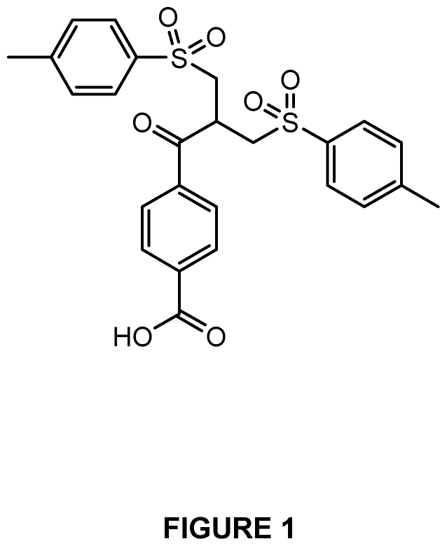

FIG. 2 shows an exemplary biochip spotting configuration used in assay experiments (see Methods).

FIG. 3 shows the synthesis of 4-[bis(4-methylphenylsulphonylmethyl)-1-oxoethyl] benzoic acid and subsequent addition of a silyl moiety (APTES) to form a substrate linking agent.

FIG. 4 shows an assay sensitivity comparison (RLU is ‘relative light unit’ value) using scFvs on ink-covered ceramic substrate (ceramic biochip) and different cross-linking agents. For each FABP protein isoform (liver, heart, adipose, epidermal, brain & testis) column to the left corresponds to sulphone substrate linking agent (see FIG. 3), and column to the right corresponds to an EPON SU-8 substrate linking agent. Lines represent CV values (n=12). Sulphone cross-linker increases assay sensitivity.

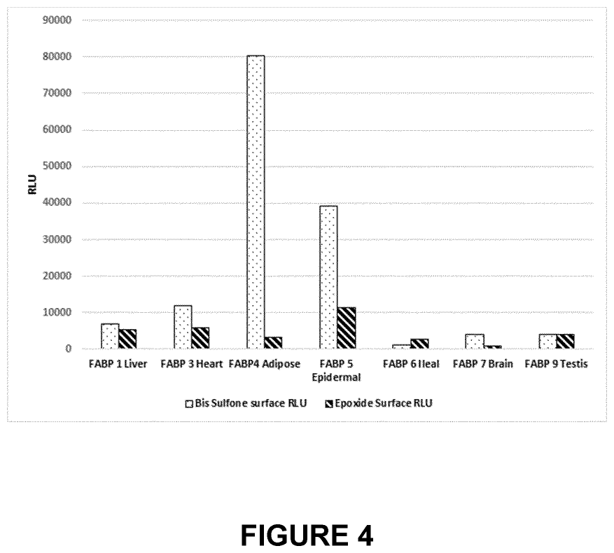

FIG. 5 shows an assay sensitivity comparison (RLU value) using scFvs and sulphone cross-linker (see FIG. 3 for structure) on ceramic substrate (ceramic biochip) with and without ink layer. For each FABP protein isoform (liver, heart, adipose, epidermal, brain & testis) column to the right corresponds to substrate with ink layer, and column to the left corresponds substrate without ink layer. Lines represent CV values (n=12). Substrate with ink layer increases assay sensitivity.

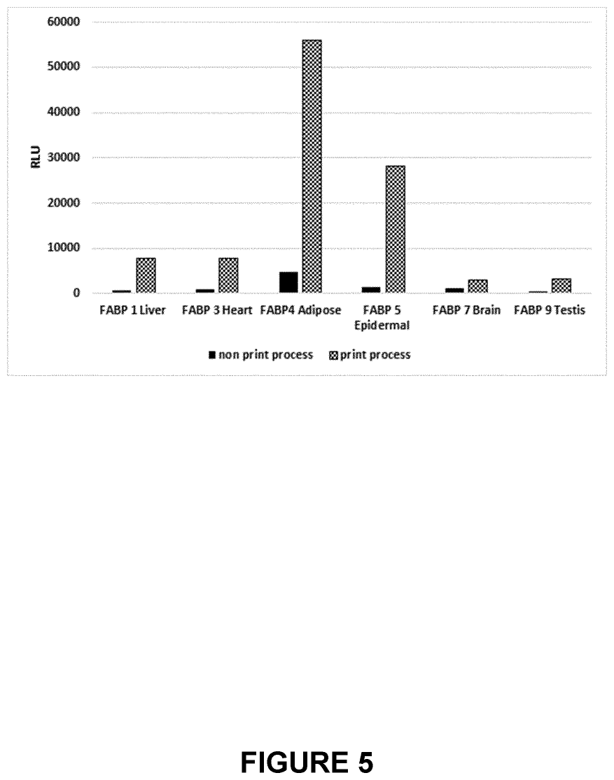

FIG. 6 shows a generic assay device components for an immunoassay incorporating a sulphone or sulphonate derivative and a binding ligand. Preferably, the binding ligand is an antibody, as described below.

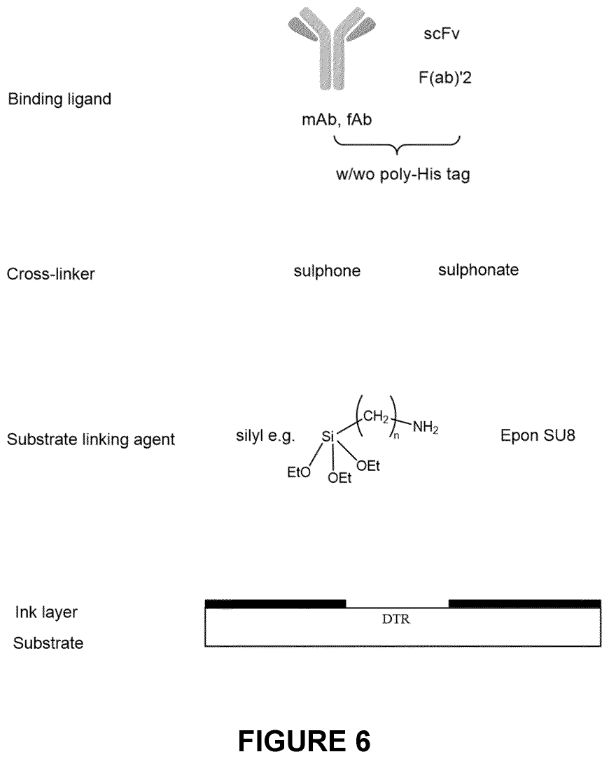

FIG. 7 shows examples of sulphone substrate linking agents.

FIG. 8 shows a comparison of different substrate linking agents using scFv binding ligands; the bound proteins were adipose (FABP 4) and epidermal (FABP 5) FABPs. The three columns represent different concentrations of FABPs. 1% Bis-sulphone corresponds to substrate linking agent B in FIG. 7, Epoly-8 is a synonym of EPON-SU8, LK2404 is substrate linking agent A in FIG. 7, LK2421 is substrate linking agent C in FIG. 7.

FIG. 9 shows a comparison of different substrate linking agents using F(ab′)₂ binding ligands; the bound proteins were adipose (FABP 4) and epidermal (FABP 4) FABPs. The three columns represent different concentrations of FABPs. 1% Bis-sulphone corresponds to substrate linking agent B in FIG. 7, Epoly-8 is a synonym of EPON-SU8, LK2404 is substrate linking agent A in FIG. 7, LK2421 is substrate linking agent C in FIG. 7.

### DETAILED DESCRIPTION

In order that the present invention may be more readily understood, certain terms are first defined. Additional definitions are set forth throughout the detailed description.

As used herein, the term “plurality of discrete reaction zones” refers to the presence of more than one discrete reaction zone present on the substrate surface, i.e. at least two discrete reaction zones.

As used herein, the terms “patient” and “subject” are used interchangeably herein and refer to any animal (e.g. mammal), including, but not limited to, humans, non-human primates, canines, felines, rodents and the like, which is to be where the sample is obtained from. Preferably, the subject or patient is a human.

As used herein, the term “a sample” includes biological samples obtained from a patient or subject, which may comprise a serum sample, plasma sample, whole blood sample, saliva sample, urine sample, mucous sample, CSF sample, sputum sample, ear wax sample, hair sample, sweat sample, meconium, skin, solid tumour extracts, peripheral blood mononuclear cells, bone marrow mononuclear cells, cerebrospinal fluid, cystic fluid, tear sample or any suitable cell lysate, preferably the sample is a serum or plasma sample.

As used herein, the term ‘detecting’ refers to qualitatively analysing for the presence or absence of a substance, for example, a signal, usually above a set threshold value to account for background signal noise, indicating presence or absence of the substance.

As used herein, the term ‘determining’ refers to quantitatively analysing for the amount of substance present.

As used herein, the term “detectably-labelled molecule” refers to a molecule that has a label covalently attached to said molecule to enable its detection. Such labels may include, but are not limited to, radionuclides, fluorophores, dyes, quantum dots, polymers or enzymes, including, for example, horse-radish peroxidase (HRP) and alkaline phosphatase. Preferably, the label is HRP.

As used herein, the term “antibody” refers to an immunoglobulin which specifically recognises an epitope on a target as determined by the binding characteristics of the immunoglobulin variable domains of the heavy and light chains (V_{H}S and V_{L}S), more specifically the complementarity-determining regions (CDRs). Many potential antibody forms are known in the art, which may include, but are not limited to, monoclonal antibodies, polyclonal antibodies, antibody fragments (for example Fab, Fab′, and Fv fragments, linear antibodies single chain antibodies and multispecific antibodies comprising antibody fragments), single-chain variable fragments (scFvs), multi-specific antibodies, chimeric antibodies, humanised antibodies and fusion proteins comprising the domains necessary for the recognition of a given epitope on a target. The various forms of antibody listed above may also be referred to as “binding ligands” or “biological molecules”. Preferably, the biological molecule, or binding ligand, of the detection assay herein disclosed is an antibody. Preferably, the detection assay herein disclosed is an immunoassay.

As used herein, the term “has affinity for”, in the context of the present invention refers to the interaction between the analyte and the biological molecule immobilised to the substrate surface. Specifically, the term “has affinity for” refers to an interaction wherein the biological molecule and analyte associate more frequently or rapidly, or with greater duration or affinity, or with any combination of the above, than when either the biological molecule or analyte is substituted for an alternative substance. Generally, but not necessarily, reference to binding means specific recognition. By way of example and not limitation, specific binding, or lack thereof, may be determined by comparative analysis with a control comprising the use of an antibody which is known in the art to specifically recognise said target and/or a control comprising the absence of, or minimal, specific recognition of said target (for example wherein the control comprises the use of a non-specific antibody). Said comparative analysis may be either qualitative or quantitative. It is understood, however, that an antibody or binding moiety which demonstrates exclusive specific recognition of a given target is said to have higher specificity for said target when compared with an antibody which, for example, specifically recognises both the target and a homologous protein.

As used herein, the term “control” refers to a value or values previously determined, determined simultaneously during the assay, or determined after the assay has been performed, to which the assay results can be assessed and quantified against. The control will typically provide a measure of the extent of binding between the biological molecule and the detectably-labelled molecule in the absence of an analyte. This can be used to provide a measure of the amount of binding that occurs in the assay between the biological molecule and analyte. The control can be established as a calibration, alternatively, a calibration curve can be generated using analyte preparations at multiple concentrations. The assay signal output generated from a sample can be applied to the calibration curve to enable quantification of the analyte level of said sample.

As used herein, the term “epitope” refers to the portion of a target which is specifically recognised by a given antibody.

As used herein, the terms “substrate linking agent”, “linker”, “linking agent” and “linking group” are used interchangeably and refers to a chemical moiety that connects the biological molecule to the substrate surface via a cross-linker.

As used herein, the term “cross-linker” refers to a sulphone-based cross linker having the structure of Formula (1), and is capable of binding a substrate linking agent to the biological molecule, i.e. it acts as the intermediatory entity between the linking agent and the biological molecule.

The present invention provides for an improved detection assay, for example, an immunoassay, whereby an analyte can be efficiently and accurately determined. Specifically, it is a surprising discovery that the use of a particular cross-linker in the detection assay i.e. a sulphone-based cross linker, produces the desirable properties herein disclosed. As such, the detection assay herein disclosed allows for an effective way in which a broad range of analytes may be efficiently and accurately determined, allowing for the use of the detection assay as an effective and rapid diagnostic tool in subjects or patients suffering from a variety of disorders/diseases.

Accordingly, in a first aspect, the present invention provides for a substrate comprising a plurality of discrete reaction zones onto which one or more biological molecule are immobilised to the substrate surface by means of a linking agent and a cross-linker, wherein the cross-linker is a sulphone-based cross linker having a structure according to Formula (1):

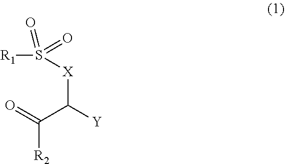

wherein X is —CH₂—,
Y is methylidene (═CH₂), or Y is selected from —CH₂—SO₂-Ph-R₄, —CH₂—SO₂-Ph-CH₃, —CH₂—SO₂—CH₃, —CH₂—O—SO₂-Ph-R₄, —CH₂—O—SO₂-Ph-CH₃ or —CH₂—O—SO₂—CH₃; preferably Y is methlylidene (═CH₂), —CH₂—SO₂-Ph-CH₃, or —CH₂—O—SO₂-Ph-CH₃, more preferably Y is methylidene (═CH₂) or —CH₂—SO₂-Ph-CH₃;
R₁ is CH₃ or CH₃-Ph, wherein the Ph is optionally substituted with one or more C₁₋₆alkyl, NO₂, F, Cl or Br; preferably R₁ is CH₃-Ph, and Ph is substituted with a C₁₋₆alkyl group, preferably methyl; and
R₂ is selected from —C₆₋₁₂aryl-Z, —C₁₋₁₈alkyl-Z, and —C₂₋₂₀alkenyl-Z, wherein Z is selected from COR₃, —NH₂ and —OH, R₃ is selected from H, OH, NH₂, —C₁₋₆alkyl-OH and (EtO)₃Si—(CH₂)ₙ—NH— in which n is 1-6; and R₄ is H or is optionally substituted with one or more C₁₋₆alkyl, NO₂, F, Cl or Br; preferably R₂ is selected from -Ph-Z, —C₁₀H₈—Z, —C₁₋₆alkyl-Z and —C₂₋₆alkenyl-Z, more preferably from -Ph-Z and —C₁₀H₈—Z, and more preferably -Ph-Z, and preferably Z is selected from COR₃.

As such, the present invention provides for a substrate suitable for a variety of assay formats, for example, the substrate disclosed herein may be used in a sandwich assay format, wherein a capture and detection agent bind to different sites on the same analyte, or in a competitive assay format, wherein the binding of the analyte to the biological molecule competes with a detectably labelled molecule for binding to the biological molecule.

The plurality of discrete reaction zones allow for spatially distinct areas on the same substrate, allowing for simultaneous detection of multiple analytes in a sample, if so desired. Accurate data may be obtained using up to approximately 1000 discrete reaction zones per 9 mm×9 mm area of substrate, however, accurate data is also obtainable at lower densities, for example, substrates having 4 discrete reaction zones per 81 mm² area of substrate.

The biological molecule(s) immobilised to the substrate surface may be any biological molecule suitable for use in a detection assay, for example, the detection of analytes in a sample. As used herein, the term “biological molecule” is used interchangeably with the term “binding ligand” in the context of the present invention. Accordingly, the chosen biological molecule immobilised to the substrate surface may be any agent that can bind to, or has affinity for, the analyte of interest, for example, biomolecules, in particular antibodies, aptamers, phages and oligonucleotides, and non-biomolecules, such as molecular imprinted polymers. In a preferred embodiment, the biological molecule immobilised to the substrate surface is a protein or polypeptide. In an even more preferred embodiment, the biological molecule immobilised to the substrate surface is an antibody. Accordingly, in one embodiment, the detection assay herein disclosed is an immunoassay.

The biological molecule is immobilised to the substrate surface via means of both a linking agent and a cross-linker. Such a configuration allows for the projection of the biological molecule away from the substrate surface, exposing a larger surface area of the biological molecule to the analyte compared to passive adsorption of the biological molecule to the substrate surface. Additionally, the interaction between the biological molecule and the linking agent/cross-linker (covalent bonding) is significantly stronger than that of the non-bonding interaction of passive adsorption. The above features results in a more sensitive assay, as well as a more robust device and method for performing said assay. The cross-linking agent can be bonded to the surface linking agent prior to the attachment to the substrate surface, alternatively, the cross-linking agent can be bonded to the substrate linking agent, the substrate linking agent having already been attached (covalently bonded) to the substrate surface or the cross-linking agent can be attached to the biological molecule prior to their attachment to the substrate linking agent.

The linking agent may be any chemical moiety that can connect the biological molecule to the substrate via a cross-linker. Preferably, the linking agent is an epoxy silane derivative, an epoxy oligomer or an epoxy polymer. Examples include, but are not limited to, (EtO)₃Si—(CH₂)n-NH₂ (see EP0874242 for silane derivative examples) and EPON-SU8 (also referred to as Epoly-8).

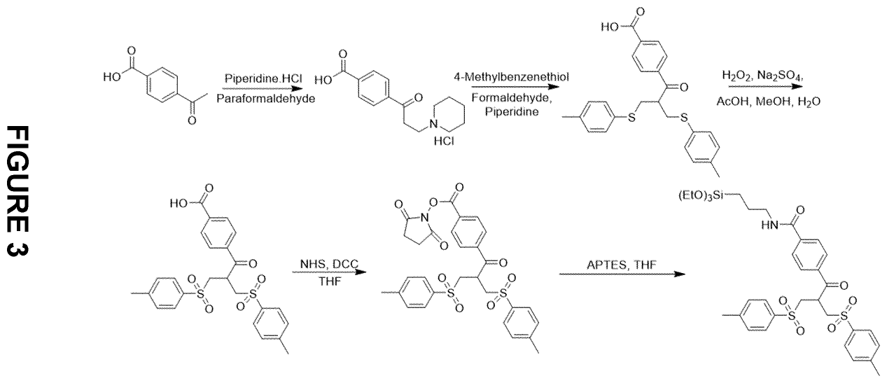

It has been surprisingly found that the use of a sulphone-based cross-linker having the structure of Formula (1) in the detection assay herein disclosed can further increase the sensitivity of the assay. “Sulphone-based cross-linker” refers to a cross-linker comprising at least one sulphone moiety. Suitable examples of sulphone-based cross-linkers include, but are not limited to: α,β-unsaturated ketone sulphones and precursors to α,β-unsaturated ketone sulphones, such as the bis-sulphone of FIG. 3. Even more preferably, α,β-unsaturated ketone sulphones and precursors to α,β-unsaturated ketone sulphones are used.

The sulphone-based cross-linker is a sulphone-based cross-linker, having the structure according to Formula (1):

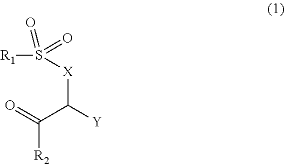

wherein X is —CH₂—;
Y is methylidene (═CH₂), or Y is selected from —CH₂—SO₂-Ph-R₄, —CH₂—SO₂-Ph-CH₃, —CH₂—SO₂—CH₃, —CH₂—O—SO₂-Ph-R₄, —CH₂—O—SO₂-Ph-CH₃ or —CH₂—O—SO₂—CH₃;
R₁ is CH₃ or CH₃-Ph, wherein the Ph is optionally substituted with one or more C₁₋₆alkyl, NO₂, F, Cl or Br; and
R₂ is selected from —C₆₋₁₂aryl-Z, —C₁₋₁₈alkyl-Z, and —C₂₋₂₀alkenyl-Z, wherein Z is selected from COR₃, —NH₂ and —OH, and R₃ is selected from H, OH, NH₂, —C₁₋₆alkyl-OH and (EtO)₃Si—(CH₂)ₙ—NH— in which n is 1-6; and
R₄ is H or is optionally substituted with one or more C₁₋₆alkyl, NO₂, F, Cl or Br.

It will be appreciated that when Y is methylidene (═CH₂) in Formula (1), a double bond is present as shown in Formula (1a) below:

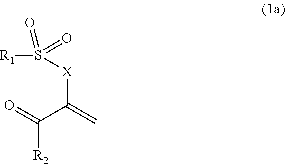

It will be appreciated that when Y is methylidene (═CH₂), the cross-linker is an α,β-unsaturated ketone sulphone, and when Y is selected from —CH₂—SO₂-Ph-CH₃, —CH₂—SO₂—CH₃, —CH₂—O—SO₂-Ph-CH₃ or —CH₂—O—SO₂—CH₃, the cross-linker is a precursor to a α,β-unsaturated ketone sulphone.

Preferably Y is methylidene (═CH₂), —CH₂—SO₂-Ph-CH₃, or —CH₂—O—SO₂-Ph-CH₃, more preferably Y is methylidene (═CH₂) or —CH₂—SO₂-Ph-CH₃.

Preferably, R₁ is CH₃-Ph, and Ph is substituted with a C₁₋₆alkyl group, preferably methyl.

Preferably, R₂ is selected from -Ph-Z, —C₁₀H₈—Z, —C₁₋₆alkyl-Z and —C₂₋₆alkenyl-Z, more preferably from -Ph-Z and —C₁₀H₈—Z, and more preferably -Ph-Z. Preferably, Z is selected from COR₃.

The sulphone-based cross-linker, preferably an α,β-unsaturated ketone sulphone or precursor thereof, may have a structure according to Formula (2):

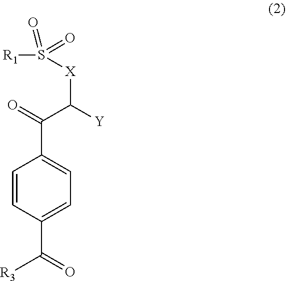

wherein X is —CH₂—,
Y is methylidene (═CH₂) or Y is selected from —CH₂—SO₂-Ph-CH₃, —CH₂—SO₂—CH₃, —CH₂—O—SO₂-Ph-CH₃ or —CH₂—O—SO₂—CH₃;
R₁ is CH₃ or CH₃-Ph, wherein the Ph is optionally substituted with one or more C₁₋₆alkyl; and
R₃ is selected from H, OH, NH₂, —C₁₋₆alkyl-OH and (EtO)₃Si—(CH₂)ₙ—NH— in which n is 1-6.

It will be appreciated that when Y is methylidene (═CH₂) in Formula 2, a double bond is present as shown in Formula (2a) below:

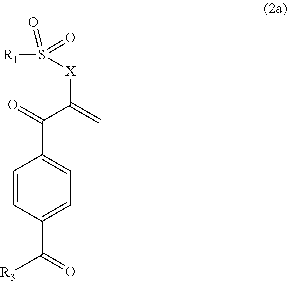

Preferably Y is methylidene (═CH₂), —CH₂—SO₂-Ph-CH₃, or —CH₂—O—SO₂-Ph-CH₃, more preferably Y is methylidene (═CH₂) or —CH₂—SO₂-Ph-CH₃.

Preferably, R₁ is CH₃-Ph, and Ph is substituted with a C₁₋₆alkyl group, preferably methyl.

Preferably, R₃ is OH, H or (EtO)₃Si—(CH₂)ₙ—NH— in which n is 1-6.

The sulphone-based cross-linker, preferably an α,β-unsaturated ketone sulphone or precursor thereof (Formula (4)), may have a structure according to Formula (3) or Formula (4):

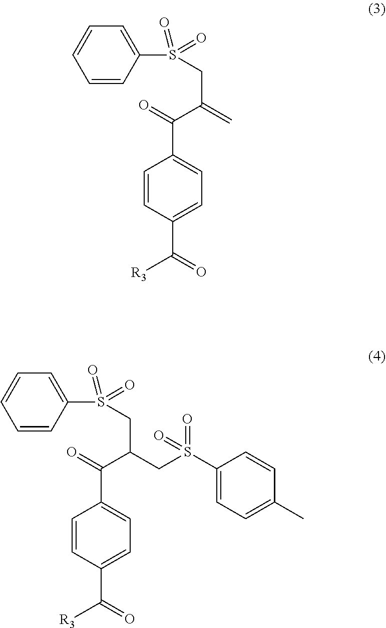

wherein R₃ is selected from H, OH, NH₂, —C₁₋₆alkyl-OH and (EtO)₃Si—(CH₂)ₙ—NH— in which n is 1-6, preferably OH, H or (EtO)₃Si—(CH₂)ₙ—NH— in which n is 1-6.

As used herein, ‘Ph’ refers to phenyl, “C₁₀H₈” refers to a naphthalene or napthyl group.

As used herein, the term “C₁₋₁₈ alkyl” denotes a straight or branched saturated alkyl group having from 1 to 18 carbon atoms; For parts of the range C₁₋₁₈ alkyl, all sub-groups thereof are contemplated, such as C₁₋₆ alkyl, C₅₋₁₅ alkyl, C₅₋₁₀ alkyl, and C₁₋₆ alkyl. Examples of said C₁₋₄ alkyl groups include methyl, ethyl, n-propyl, iso-propyl, n-butyl, iso-butyl, sec-butyl, and tert-butyl. The alkyl groups may be optionally substituted with one or more functional groups, including C₁₋₁₈ alkyl groups, “C₆₋₁₂ aryl”, and “C₁₋₁₈ alkoxy”, halogen, and “C₃₋₁₈ cycloalkyl”.

As used herein, the term “C₂₋₁₈alkenyl” denotes a “C₁₋₁₈ alkyl” group containing some degree of unsaturation (partial unsaturation) i.e. containing one or more alkene/alkenyl moiety(s).

As used herein, the term “C₆₋₁₂ aryl” denotes a monocyclic or polycyclic conjugated unsaturated ring system having from 6 to 12 carbon atoms. For parts of the range C₆₋₁₂ aryl, all sub-groups thereof are contemplated, such as C₆₋₁₀ aryl, C₁₀₋₁₂ aryl, and C₆₋₈ aryl. An aryl group includes condensed ring groups such as monocyclic ring groups, or bicyclic ring groups. Examples of C₆₋₁₂ aryl groups include phenyl, biphenyl, indenyl, naphthyl or azulenyl. Condensed rings such as indane and tetrahydro naphthalene are also included in the C₆₋₁₂ aryl group. The aryl groups may be optionally substituted with other functional groups. The aryl groups may be optionally substituted with one or more functional groups, including C₁₋₁₈ alkyl groups, halogen, and “C₁₋₁₈ alkoxy”. The aryl groups may be substituted with these substituents at a single position on their unsaturated ring system, or may be substituted with these substituents at multiple positions on their unsaturated ring system.

The terms “unsaturated” and “partially saturated” refer to rings wherein the ring structure(s) contains atoms sharing more than one valence bond i.e. the ring contains at least one multiple bond e.g. a C═C, C≡C or N═C bond. The term “fully saturated” refers to rings where there are no multiple bonds between ring atoms.

“Optional” or “optionally” means that the subsequently described event or circumstance may but need not occur, and that the description includes instances where the event or circumstance occurs and instances in which it does not.

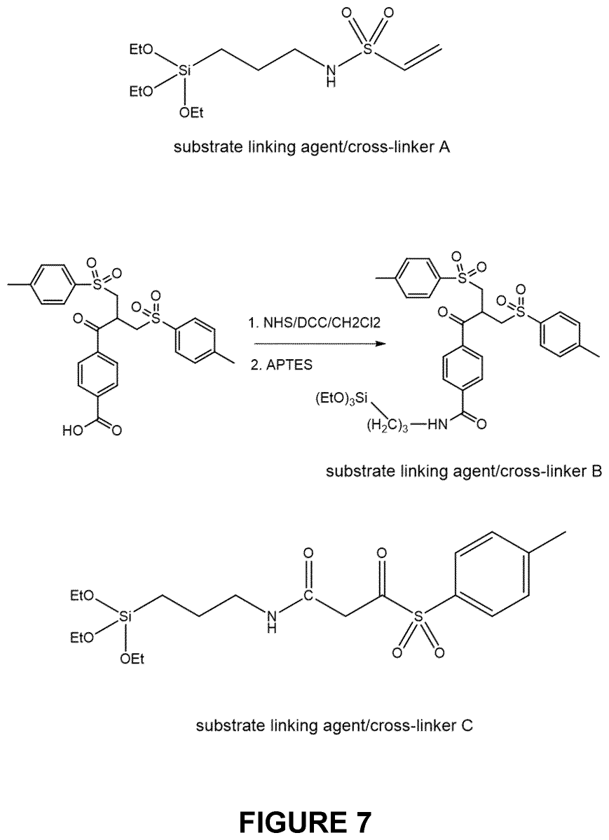

In the context of the present invention, the cross-linker may be subject to chemical activation prior to attachment to the substrate linking agent. By ‘chemical activation’ is meant a process whereby the cross-linker is altered such that it has increased propensity for subsequent reaction, i.e. increased propensity for bonding with the substrate linking group and/or biological molecule. Suitable methods of chemical activation will be well known by a skilled person, for example, the EDC method or a maleimido-group incorporation. For example, when Z of formula (1) is COR₃ and R₃ is OH, or R₃ of formula (2) is OH, the EDC method or a maleimido-group incorporation may be used to activate the cross-linker. It will be understood by a skilled person that such chemical activation is typically used to promote bonding of the cross-linker to the substrate linking group and/or the biological molecule. Accordingly, the present method also provides for the incorporation of a substrate linking agent in the sulphone-based cross-linker prior to the addition to the substrate. This has the advantage of omitting the substrate chemical-activation step, which in turn benefits efficiency of device production. FIGS. 3 and 7 present examples of silyl-sulphone compounds.

The detection assay herein described allows for the accurate and efficient detection and measurement of a broad range of analytes. Accordingly, the detection assay herein described may be used as a diagnostic assay in diagnosing a variety of diseases, disorders or infections. Alternatively, the detection assay herein described be used as a predictive assay, for example, to determine if a patient may benefit from a specific treatment, or be used as a prognostic assay. For example, the detection assay may be used to detect analytes in a sample known to be associated with cancer, neurodegenerative disease, kidney disease, cardiovascular disease, respiratory disease, gastrointestinal disease, liver disease, central nervous system disease, viral infections or bacterial infections. The detection assay herein disclosed may be used for single analyte detection or multiple analyte detection depending on the purpose of said assay. It is understood that the biological molecule immobilised to the substrate surface will be dependent on the purpose of said assay and specific to the analyte whose detection is desired. The biological molecule may be a biomolecule, for example, antibodies, phages and oligonucleotides, or a non-biomolecule, for example, molecular imprinted polymers. In a preferred embodiment, the biological molecule is a biomolecule. In a more preferred embodiment, the biological molecule is a protein or a polypeptide. For example, the biological molecule may be a protein, for example a viral protein, for which the analyte is known to display a level of specificity for, for example, an antibody. In some instances said viral protein may be a coronavirus protein, for example, the spike protein, the nucleocapsid protein, the membrane protein or the envelope protein of SARS-CoV, SARS-CoV2 or MERS-CoV. In a preferred embodiment, the binding protein is a viral protein that is not a coronavirus protein, for example, the binding protein is not a coronavirus spike protein, the binding protein is not a coronavirus nucleocapsid protein, the binding protein is not a coronavirus membrane protein, the binding protein is not a coronavirus envelope protein of SARS-CoV, SARS-CoV2 or MERS-CoV.

In a further embodiment the biological molecule is a non-viral protein or peptide.

In yet a more preferred embodiment, the biological molecule is an antibody, for example a polyclonal antibody, a monoclonal antibody, a short chain variable fragment (scFv), a variable antibody domain (F(ab′)₂), a single domain antibody (sdAb), a bispecific antibody, a fusion protein or any combination thereof. In a most preferred embodiment, the biological molecule is a scFv or F(ab′)₂ biological molecule, for example, a FABP scFv or FABP F(ab′)₂, more specifically, the biological molecule may be a FABP protein isoform 1, 3, 4, 5, 6, 7 or 9 scFv or F(ab′)₂.

The biological molecule may bond to the substrate linking agent and cross-linker through inherent functional groups such as carboxy, amino or sulphur present in the amino acids that constitute the protein. However, the assay effectiveness may be improved via the incorporation of a chemical activating group in the biological molecule. The attachment of the biological molecule to a substrate linking agent and cross-linker requires that the binding characteristics of the biological molecule are not affected, and that the specificity and affinity of the biological molecule following the bonding process remain fit for purpose. Possible deleterious effects upon the biological molecule upon chemical activation and or bonding to the substrate include biological molecule tertiary structure disruption leading to reduced bonding and/or specificity to the target analyte. Accordingly, the method herein disclosed may use a biological molecule immobilised to the substrate surface that further comprises a protein tag. Preferably, the method herein disclosed may use a biological molecule immobilised to the substrate surface that further comprises a poly-His tag or a biological molecule immobilised to the substrate surface that has been modified to have a poly-His tag. Accordingly, such a modification may result in a recombinant biological molecule that helps to reduce structural disruption of the biological molecule when bound to the linking agent and cross-linker. Additionally, it will be readily understood that such a modification, i.e. the inclusion of protein tag, preferably a poly-His tag, may also be used for recombinant protein purification. The skilled person will readily understand the methods by which a poly-His tag could be incorporated into a biological molecule, for example, recombinant DNA methods comprising inserting the DNA encoding a protein into a suitable vector encoding a His-tag (Loughran and Walls, (2011), Purification of poly-histidine-tagged proteins, Methods Mol Biol, 681:311-35).

Therefore, the method herein disclosed may comprise a recombinantly-produced biological molecule incorporating a poly-His tag attached to a substrate linking agent and cross-linker. The poly-His tag can be varied in the number of histidine units but it is preferable to incorporate between 2 and 10 histidines, more preferably 6, 7 or 8 histidines; a 6 unit histidine tag is most preferred.

The substrate of the detection assay herein disclosed may further comprise a coating of a masking material, wherein the discrete reaction zones are uncoated areas on the substrate and wherein the masking material comprises an acrylic resin and has a water contact angle of 60-120°. Use of a coated substrate in the context of the present invention provides for an improved detection sensitivity and image quality, especially when using digital sensors. The coated areas of the substrate reduce non-specific binding and the cross-linking of the spots and so background noise is reduced resulting in an enhanced signal-to-noise ratio. This means that the signal obtained is proportional to the extent of the “true” binding that has occurred between the sample and the targets on the substrate. Increased spatial resolution is also achieved.

Preferably the coating is a non-silicon containing coating and preferably lacks elemental silicon or a compound incorporating silicon. Silicon-containing coatings are known in the prior art, for example, silcone. However the inventors have found that when using such coatings, silicon can contaminate the discrete reaction zones, which has a detrimental effect on attachment of the biological molecule.

Any suitable non-silicon masking or coating may be used. Preferably the coating comprises one or more resins selected from the list of acrylics, alkyds, epoxides, hydrocarbons, phenolics or fluoropolymers such as a polytetrafluoroethylene (PTFE). The coating may also contain any suitable ink solvents and/or ink additives. Particularly preferred ink solvents include cyclohexanone, butoxyethanol and aromatic distillates. Particularly preferred ink additives include carbon black (black pigment), mineral oil (wetting agent), petroleum distillate, dibutyl phthalate (plasticiser), salts of cobalt, manganese or zirconium (drying agent), aluminium and titanium chelator (chelating agent), antioxidants, surfactants and defoamers.

Preferably the coating (masking material) comprises an acrylic resin, a pigment and a structuring agent. One pigment may be present or multiple pigments may be used. Acrylic resins are used to increase ink viscosity, rheological properties and adhesion to the substrate. The pigment imparts a dark colour, preferably a black colour, and hence imparts optical opacity to the ink. The structuring agent provides hydrophilic/hydrophobic properties to the surface of the substrate and also help adhesion to the substrate.

Preferably the pigment is present in an amount of 1 to 15% w/w of the masking composition (masking material); the acrylic resin is present in an amount of 1 to 20% w/w, and the structuring agent is present in an amount of 10 to 60% w/w.

More preferably, the pigment, preferably black pigment, is present in an amount of 1 to 8% w/w of the masking composition; the acrylic resin is present in an amount of 2 to 15% w/w of the masking composition, and the structuring agent is present in an amount of 15-50% w/w of the masking composition.

Most preferably, the pigment, preferably black pigment, is present in an amount of 5% w/w of the masking composition; the acrylic resin is present in an amount of 10% w/w of the masking composition, and the structuring agent is present in an amount of 20% w/w of the masking composition

In a preferred embodiment, carbon black pigment is used in the masking material, preferably Elftex 285. Preferably the acrylic resin is B-67. Preferably the structuring agent is a PTFE wax, such as CERAFLOUR® 965.

Most preferably, the pigment is Elftex 285, the acrylic resin is B-67 and the structuring agent is CERAFLOUR® 965.

The coating (masking material) may further comprise one or more agents selected from the list of solvents, such as ethanol, propanol, xylene, diglycol, butyl ether; dispersing agents; pigment wetting agents; levelling agents; pigment wetting agents and/or crosslinking agents.

Preferably, the coating has a contact angle of 20-175°, more preferably 20-170° more preferably 90-120°, even more preferably about 110°. The measurement is taken using the following protocol: The contact angle is measured using a KSV CAM200 contact angle meter equipped with automated dispenser controlled using stepper motor, LED source and CCD camera. The contact angle meter is connected to a software tool for dispense controller, image grabbing and image analysis. A droplet of deionised water of 3.5 μl is dispensed on the substrate at a predefined location and the image is captured using a CCD camera. Image analysis is performed using software to estimate the contact angle of the water droplet.

Preferably, the thickness of the coating applied to the substrate is 1-100 μm thick, preferably, 2-50 μm thick. This creates a discrete reaction zone that is a well having a depth of 1-100 μm, preferably 2-50 μm, respectively. Most preferably the thickness of coating is 3 to 20 μm thick μm and the resulting depth of the well is 3 to 20 μm in depth.

The walls of the discrete reaction zones, or wells, are formed by the surrounding coating. When a buffer solution containing a biological molecule is spotted onto the discrete reaction zones, the binding agent is immobilised on the activated surface. The walls of each discrete reaction zone can absorb scattering light leading to improved data readings.

Preferably, the coating is darker than the substrate. This will provide contrast between the discrete reaction zones and the surrounding coated area. More preferably, the substrate is white and the coating is any colour ranging from off-white to black. Any colour other than white will provide contrast between the discrete reaction zones and the surrounding coated area. The contrast between the discrete reaction zones and the surrounding area of coated substrate gives better spatial resolution and allows accurate data even with a high density of discrete reaction zones. The term “masking” material herein means any material used to coat the substrate, preferably having a colour darker than the substrate. Preferably the masking material has a colour ranging from off-white to black. More preferably, the masking material has a matt finish.

The coating (masking material) can be made using techniques known to the person skilled in the art.

The present invention makes use of conventional apparatus to accurately image arrayed molecules. Preferably, the coating is applied using screen print techniques known to the person skilled in the art. The substrates are screen printed to provide discrete reaction zones that are uncoated regions on the substrate.

Preferably the only areas of the substrate not coated are the discrete reaction zones. This makes it possible for robotic software to physically locate colour-contrasted/geometrically discrete features and accurately deposit biological molecules in a specific location. This is specifically important with increasing density of the discrete reaction zones which may increase the overall risk of rejections of the biochip based on the x and y coordinates of reaction zones.

The substrate itself may comprise any suitable material known to the skilled person. Preferably the substrate comprises silicon, metal oxides, ceramic, glass or plastic. More preferably, the substrate is a ceramic, glass or plastic substrate. More preferably the ceramic is aluminium oxide based. Most preferably the substrate is a white ceramic substrate. The latter gives the most contrast between the substrate and the coating of a masking material that is applied to the substrate.

A ceramic substrate may be manufactured to provide a range of grain sizes (1 to 30 μm). The preferred particle size of the ceramic substrate used in this invention is less than 20 μm, preferably less than 10 μm. The reduced particle size imparts much improved surface uniformity which in turn provides enhanced performance of biological assays.

The preferred ceramic material consists of about 96% alumina (Al₂O₃) with a particle size in the range of 4-8 μm. The material is vacuum-tight, and has a surface topography of 0.6 to 0.8 μm when ground. The surface uniformity can be improved by a polishing process, to yield a surface with variation of 0.4-0.5 μm. A further improvement is achieved by lapping and polishing, to yield a surface with a variability of 0.05-0.1 μm.

The substrate for use in the present invention may be of a planar conformation, such as a glass slide, microtitre plate or a chip/biochip. As used herein, the term “biochip” refers to a chip whose use is biomedical and is made of a thin, wafer-like substrate with a planar surface. Preferably, the substrate may be a bio-chip due to its stability and adaptability. Whist the biochip may be made of any suitable material, such as glass or any suitable polymer, preferably, the biochip is made of ceramic. Even more preferably, the biochip is made of aluminium oxide-based ceramic and may be chemically activated. Various aspects of biochip technology are described in EP0874242.

In a second aspect, the present invention provides for a substrate having a biological molecule immobilised thereof, comprising attaching the biological molecule to the substrate surface by means of a sulphone-cross-linker having a structure according to Formula (1). The substrate may have on its surface a single biological molecule immobilised to the substrate surface, or may have multiple different types, i.e. scFvs and F(ab′)₂ of biological molecule, each type capable of binding a different analyte in a sample.

In a third aspect, the present invention provides for a method of detecting the presence of an analyte obtained from a subject, wherein said analyte has affinity for a biological molecule, or portion thereof, and wherein said method comprises bringing the sample obtained from the subject into contact with the biological molecule, or portion thereof, which is immobilised on a substrate support, detecting the binding of the analyte to the biological molecule, or portion thereof, via means of a detectably-labelled molecule, and measuring the amount of binding of said analyte compared to a control, wherein the biological molecule, or portion thereof, is immobilised to the substrate surface by means of a linking agent and a cross-linker, wherein the cross-linker is a sulphone-based cross-linker having a structure according to Formula (1).

The method herein provides for a detection assay in which an analyte of interest in a sample and a detectably-labelled molecule can be used to determine the level of binding to the biological molecule connected to the substrate surface via the linking agent and the cross-linker described above. Accordingly, the detectably-labelled molecule provides a detectable and measurable signal by which the presence of an analyte can be detected and/or the amount of analyte present in a sample quantified.

The measurable signal may be electromagnetic radiation based on, for example, phosphorescence, fluorescence, chemiluminescence (e.g. HRP/luminol/peroxide system). Preferably, fluorescence or chemiluminescence is used. An example of a detecting agent suitable for use with the present invention is the streptavidin-biotin-enzyme complex, avidin may also be used in place of streptavidin, resulting in a complex with the molecule to be detected. Accordingly, the method herein disclosed may include a detectably-labelled molecule labelled with a streptavidin-biotin-enzyme complex or an avidin-biotin-enzyme complex. Preferably, the enzyme of said complex may be HRP, which when exposed to luminol/peroxide system produces a detectable signal. A calibrator or standard, which can be used for effecting assay calibration, is well known in the art and enables a threshold concentration or the exact or calibrator equivalent amount of analyte(s) to be determined. The determination of an exact or calibrator equivalent amount of analyte(s) usually requires the construction of a calibration curve (also known as a standard curve). The number of calibrator points vary but is preferably from 5 to 9. Alternatively, the calibrator value can be a single pre-determined threshold value.

The sample for use in the method of the present invention may be any biological sample taken from the individual in which an analyte of interest may be detected. Preferably, the biological sample(s) require no pre-processing and can be used neat in the assay herein disclosed. Accordingly, the sample may be a serum sample, plasma sample, whole blood sample, urine sample, mucous sample, saliva sample, CSF sample, sputum sample, ear wax sample, hair sample, sweat sample, tear sample, meconium, skin, solid tumour extracts, peripheral blood mononuclear cells, bone marrow mononuclear cells, cerebrospinal fluid, cystic fluid or any suitable cell lysate. Preferably, the sample is a serum or plasma sample; alternatively, it is a whole blood sample or a saliva sample. The sample may be obtained from the subject or patient by methods routinely used in the art, for example, via venous blood collection, swab testing or tissue biopsy. The determination and/or detection of analytes, for example, antibodies may be done on one or more samples obtained from the subject. The analyte may be a protein. In one preferred embodiment, the analyte is an antibody.

The invention is further described with reference to the following non-limiting examples:

### EXAMPLES

### Example 1

### Substrate Preparation

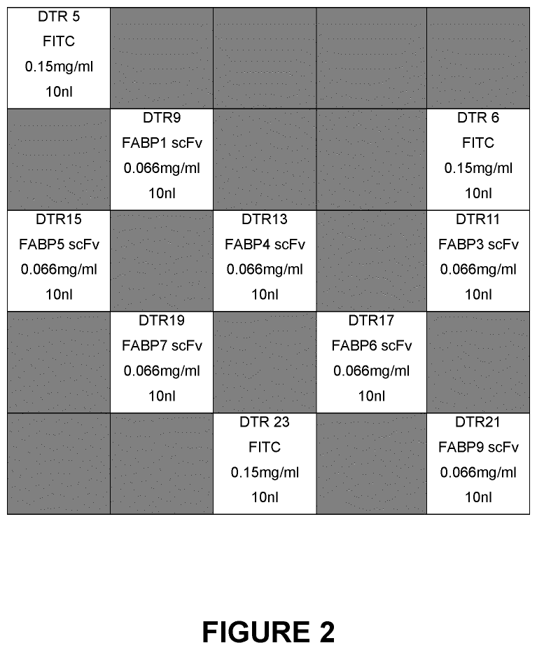

A ceramic substrate was washed with RBS 35 concentrate and water and plasma treated before addition of EPON SU8 (Hexion Incorporated, Ohio, US) or (OEt)₃-Si—(CH₂)₃—NH₂, or a silyl-sulphone (B of FIG. 7). Following stirring it was deposited on the ceramic substrate by spray coating and the chips cured for 1 hr at 140° C. For ink formulations and preparation of ink-coated chips see WO2017085509. Various ink formulations can be used on the substrate as described in WO2017085509; the preferred ink substrate comprises a carbon black pigment, a PTFE structuring agent and acrylic resin. 10 nl of scFv binding ligand incorporating a poly-His tag in carbonate/bicarbonate buffer pH 9.5 was spotted onto discrete test regions on the respective substrates using a sciFLEXARRAYER S100. FITC controls were spotted at 0.15 mg/ml in the same buffer onto discrete DTRs (×3). The substrate was left to incubate at 37° C. for 24 hr prior to use. After these steps, substrates were assembled into carriers of n=9 and analysed using an Evidence Investigator. FIG. 2 shows the biochip substrate configuration for spotted scFv.

### Example 2

### Substrate Linking Agent/Cross-Linker Preparation (See FIGS. 3 & 7)

Substrate linking agent/Cross-linker A. To a cooled solution (0° C.) of (3-aminopropyl) triethoxysilane (APTES) (22.131 g, 0.1 mol) and diisopropylethylamine (20.9 mls, 0.12 mol) in dichloromethane (300 ml) under nitrogen was added dropwise a solution 2-chloroethylsulphonyl chloride (16.63 g, 0.102 mol) in dichloromethane (50 mls). The mixture was than stirred at 0° C. for two hours and then overnight at room temperature. The solution was washed (150 ml) by water and brine (100 mL). The solution was dried over sodium sulphate filtered and concentrated to dryness. The crude product obtained was purified by flash chromatography on silica gel using ethyl acetate/hexane (40/60) to give a clear oil in 80% yield.

Substrate linking agent/Cross-linker B. To a solution of bis-sulphone acid (2 g, 4 mmol) in dichloromethane was added N-hydroxysuccinimide (506 mg, 1.1 eq) and dicyclohexylcarbodiimide (908 mg, 1.1 eq) under nitrogen. The mixture was stirred at room temperature for 2 h, the reaction was filtered to remove the urea by-product and the filtrate was evaporated to dryness in vacuo to give the crude product (2.74 g) as a white solid. The crude bis-sulphone acid N-hydroxysuccininide (2.74 g, 4.59 mmol) was dissolved in anhydrous tetrahydrofuran (50 ml) under nitrogen. To this was added APTES (1.016 g, 1 eq) and the reaction stirred at room temperature overnight. Solvents were removed in vacuo and the crude purified by flash chromatography on silica gel using 20-50% ethyl acetate in pet ether to give the title compound (2.08 g, 74%) as a pale-yellow viscous oil/semi-solid.

Substrate linking agent/Cross-linker C. Monosulphone carboxylic acid (2.4027 g, 10 mmol) was dissolved in dichloromethane (40 ml) containing N,N-dimethylformamide (1 ml). To the suspension was added oxalyl chloride (5 ml, 5.8 eq) in dichloromethane (10 ml) dropwise. On completion of addition the mixture was stirred at room temperature for 1 h. The solvents were removed in vacuo to give the crude product (2.62 g) as a solid. The crude acid chloride (2.62 g, 10 mmol) was dissolved in anhydrous tetrahydrofuran (50 ml) under nitrogen. To this was added APTES (2.21 g, 1 eq) and triethylamine (2.02 g, 2 eq) and the reaction stirred at room temperature for 2 h. Dichloromethane (100 ml) was added and washed with brine (50 ml). Organics were dried over sodium sulphate, filtered and evaporated to dryness. The crude was purified by flash chromatography on silica gel using 50% ethyl acetate in dichloromethane to give the title compound (1.948 g, 45%) as a yellow viscous oil.

### Example 3

### Evidence Investigator Analysis

288 μl of Assay diluent (EV808), then 12 μl of neat sample/neat controls were added to the appropriate biochip wells. Biochips, prepared in Example 2 using EPON SU8 as the substrate linking agent, were incubated for 30 min at 37° C. in a thermoshaker at 370 rpm. The biochips were then washed with TBST wash buffer (BT020/000/UL, Randox)—2 washes followed by 4 washes at 2 min intervals. 300 μl of FABP-HRP conjugate was added to the appropriate discrete test region (DTR). The biochips were incubated again for 30 min at 37° C. in a thermoshaker at 370 rpm followed by washing with TBST buffer (BT020/000/UL, Randox—2 washes followed by 4 washes at 2 min intervals). The biochips were developed with 250 μl of a 1:1 ratio of luminol:peroxide (EV841, Randox) for 2 min in the dark and then imaged on the Randox Evidence Investigator.

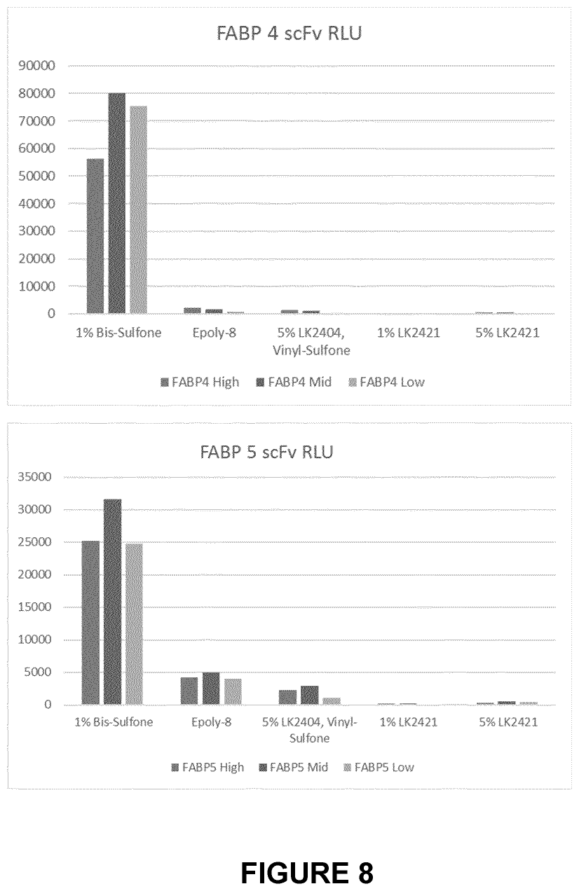

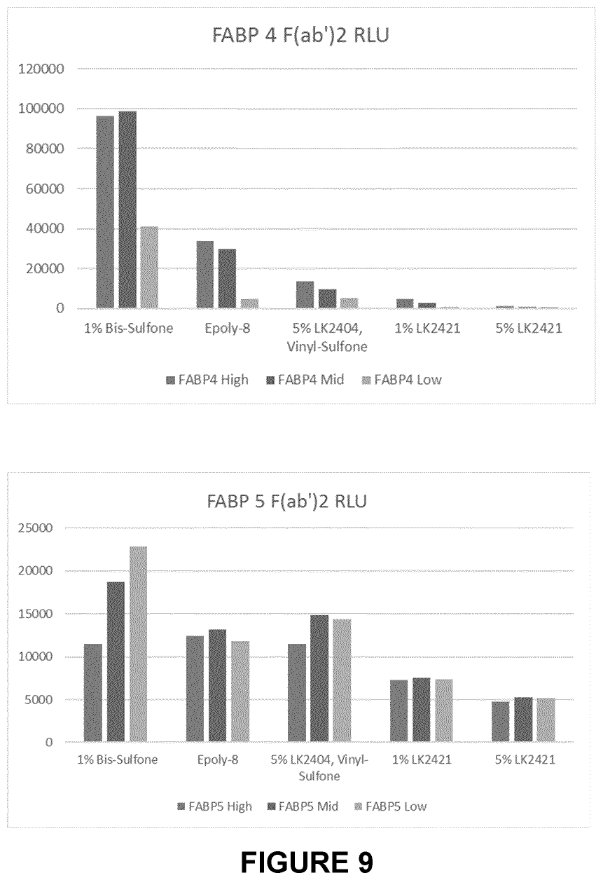

The scFV and F(ab′)₂ fragments binding ligands attached via the various substrate linking agents/cross-linking groups produced positive protein detection results without the need of a sample pre-incubation step. Improved detection sensitivity was achieved using Epon-SU8 or an αβ-unsaturated keto-sulphone derivative; unsaturated sulphones without the as configurations produced less sensitive assays than αβ-unsaturated keto-sulphones (see FIGS. 8 and 9).

1. A substrate comprising a plurality of discrete reaction zones onto which one or more biological molecules are immobilised to the substrate surface by means of a linking agent and a cross-linker, wherein the cross-linker is a sulphone-based cross-linker having a structure according to Formula (1): 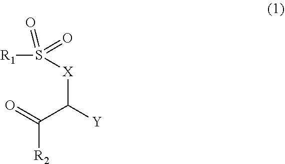
wherein X is —CH₂—;
Y is methylidene (═CH₂), or Y is selected from —CH₂—SO₂-Ph-R₄, —CH₂—SO₂-Ph-CH₃, —CH₂—SO₂—CH₃, —CH₂—O—SO₂-Ph-R₄, —CH₂—O—SO₂-Ph-CH₃ or —CH₂—O—SO₂—CH₃;
R₁ is CH₃ or CH₃-Ph, wherein the Ph is optionally substituted with one or more C₁₋₆ alkyl, NO₂, F, Cl or Br; and
R₂ is selected from —C₆₋₁₂aryl-Z, —C₁₋₁₈alkyl-Z, and —C₂₋₂₀alkenyl-Z, wherein Z is selected from COR₃, —NH₂ and —OH, R₃ is selected from H, OH, NH₂, —C₁₋₆alkyl-OH and (EtO)₃Si—(CH₂)ₙ—NH— in which n is 1-6; and R₄ is H or is optionally substituted with one or more C₁₋₆alkyl, NO₂, F, Cl or Br.

2. The substrate of claim 1, wherein the linking agent is an epoxy silane derivative, an epoxy oligomer or, an epoxy polymer.

3. The substrate of claim 1, wherein the cross-linker is a sulphone-based cross-linker and has a structure according to Formula (2): 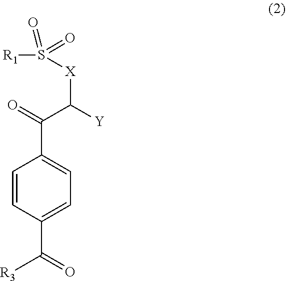
wherein X is —CH₂—;
Y is methylidene (═CH₂) or Y is selected from —CH₂—SO₂-Ph-CH₃, —CH₂—SO₂—CH₃, —CH₂—O—SO₂-Ph-CH₃ or —CH₂—O—SO₂—CH₃;
R₁ is CH₃ or CH₃-Ph, wherein the Ph is optionally substituted with one or more C₁₋₆alkyl; and
R₃ is selected from H, OH, NH₂, —C₁₋₆alkyl-OH and (EtO)₃Si—(CH₂)ₙ—NH— in which n is 1-6.

4. The substrate according to claim 1, wherein the cross-linker is a sulphone-based cross-linker and has a structure according to Formula (3) or Formula (4): 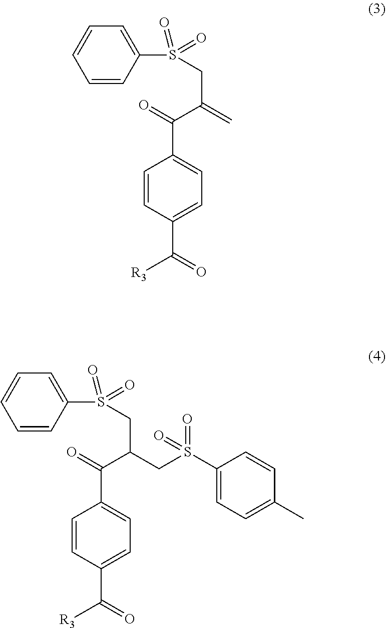
wherein R₃ is selected from H, OH, NH₂, —C₁₋₆alkyl-OH and (EtO)₃Si—(CH₂)ₙ—NH— in which n is 1-6.

5. The substrate of claim 1, wherein the biological molecule is a protein or a polypeptide.

6. The substrate of claim 1, wherein the biological molecule is a polyclonal antibody, a monoclonal antibody, a short chain variable fragment (scFv), a variable antibody domain (F(ab′)₂), a single domain antibody (sdAb), a bispecific antibody, a fusion protein or any combination thereof.

7. The substrate of claim 1, wherein the biological molecule further comprises a protein tag, preferably wherein the protein tag is a poly-His tag.

8. The substrate of claim 1, wherein the substrate further comprises a coating of a masking material, wherein the discrete reaction zones are uncoated areas on the substrate and wherein the masking material comprises an acrylic resin and has a water contact angle of 60-120°.

9. The substrate of claim 8, wherein the masking material further comprises a pigment.

10. The substrate of claim 1, wherein the substrate comprises silicon, metal oxides, ceramic, glass or plastic.

11. The substrate of claim 1, wherein the substrate is a bio-chip.

12. A method for producing a substrate having a biological molecule immobilised thereon, comprising attaching the biological molecule to the substrate surface by means of a sulphone-based cross-linker having a structure according to Formula (1).

13. A method of detecting the presence of an analyte in a sample obtained from a subject, wherein said analyte has affinity for a biological molecule, or portion thereof, and wherein said method comprises bringing the sample obtained from the subject into contact with the biological molecule, or portion thereof, which is immobilised on a substrate support, detecting the binding of the analyte to the biological molecule, or portion thereof, via means of a detectably-labelled molecule, and measuring the amount of binding of said analyte compared to a control, wherein the biological molecule, or portion thereof, is immobilised to the substrate surface by means of a linking agent and a cross-linker, wherein the cross-linker is a sulphone-based cross-linker having a structure according to Formula (1).

14. The method of claim 12 or 13, wherein the substrate comprises a Plurality of discrete reaction zones onto which one or more biological molecules are immobilised to the substrate surface by means of a linking agent and a cross-linker, wherein the cross-linker is a sulphone-based cross-linker having a structure according to Formula (1): 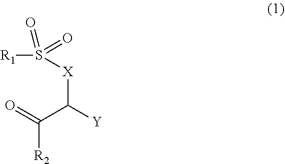
wherein X is —CH₂—;
Y is methylidene (═CH₂), or Y is selected from —CH₂—SO₂-Ph-R₄, —CH₂—SO₂-Ph-CH₃, —CH₂—SO₂—CH₃, —CH₂—O—SO₂-Ph-R₄, —CH₂—O—SO₂-Ph-CH₃ or —CH₂—O—SO₂—CH₃;
R₁ is CH₃ or CH₃-Ph, wherein the Ph is optionally substituted with one or more C₁₋₆ alkyl, NO₂, F, Cl or Br; and
R₂ is selected from —C₆₋₁₂aryl-Z, —C₁₋₁₈alkyl-Z, and —C₂₋₂₀alkenyl-Z, wherein Z is selected from COR₃, —NH₂ and —OH, R₃ is selected from H, OH, NH₂, —C₁₋₆alkyl-OH and (EtO)₃Si—(CH₂)ₙ—NH— in which n is 1-6; and R₄ is H or is optionally substituted with one or more C₁₋₆alkyl, NO₂, F, Cl or Br.

15. The method of claim 14, wherein the sample is a serum sample, plasma sample, whole blood sample, saliva sample, urine sample, mucous sample, CSF sample, sputum sample, ear wax sample, hair sample, sweat sample, meconium, skin, solid tumour extracts, peripheral blood mononuclear cells, bone marrow mononuclear cells, cerebrospinal fluid, cystic fluid, tear sample or any suitable cell lysate, preferably the sample is a serum or plasma sample.

16. The method of claim 15, wherein the detectably-labelled molecule is labelled with horseradish peroxidase (HRP), a streptavidin-biotin enzyme complex or an avidin-biotin enzyme complex.

17. The method of claim 16, wherein the analyte is a protein.

18. The method of claim 17, wherein the analyte is an antibody.
